(19) United States
(12) Patent Application Publication
(10) Pub. No.: US 2005/0113911 A1
(43) Pub. Date: May 26, 2005

(54) Adjustable intraocular lens for insertion into the capsular bag

(76) Inventor: Gholam A. Peyman, New Orleans, LA (US)

Correspondence Address: BELL, BOYD, & LLOYD LLC, P. O. BOX 1135, CHICAGO, IL, 60690-1135, US

(21) Appl. No.: 10/958,826

(22) Filed: Oct. 4, 2004

Related U.S. Application Data
(63) Continuation-in-part of application No. 10/272,402, filed on Oct. 17, 2002.

(51) Int. Cl. A61F 2/16

(52) U.S. Cl. 623/6.11

(57) ABSTRACT

The present invention relates to an intraocular lens, including a flexible capsule adapted to be inserted into the natural lens capsular bag. A polymerized portion is positioned within the flexible capsule, and an unpolymerized material is located within the flexible capsule, and has loose monomers and a polymerization initiator so that the unpolymerized material changes its volume when exposed to an energy source.

### FIELD OF THE INVENTION

The present invention generally relates to a method of inserting an intraocular lens in an eye. More specifically, the present invention relates to a method of replacing a crystalline lens in an eye with an artificial liquid or partially liquid intraocular lens.

### BACKGROUND OF THE INVENTION

An eye can have various disorders which affect the crystalline lens of the eye. One of the most common disorders is cataracts, which is a clouding of the crystalline lens. The conventional treatment for cataracts is removal of the crystalline lens and replacement of the lens with an artificial or intraocular lens (IOL).

Once an IOL is implanted, however, it generally has a fixed refractive power. This presents a problem with respect to both far and near vision. With respect to far vision, the diopter power of the IOL is generally not capable of perfect vision—i.e. 20/20. This problem is due to the fact that the refractive power of the IOL must be chosen prior to implantation and thus can only be approximated. Since the diopter power can only be approximated, most patients will require at least a ±1.00 diopter power correction along the optical path to provide perfect vision. With respect to near vision, an artificial lens results in a loss of accommodation (i.e., the process of focusing the eye between far objects and near objects).

In an attempt to avoid loss of accommodation, a technique has been developed that involves removing the crystalline lens and leaving the capsular bag that holds the crystalline lens substantially intact. Once the lens has been removed, a new lens is created in situ by filling the capsular bag with a liquid material and polymerizing or curing the liquid to form an IOL in situ. The newly formed lens has characteristics that approximate the function of a crystalline lens. By leaving the capsular bag substantially intact, the newly formed IOL will be able to focus the eye between near and far objects better than if the capsular bag is removed since the capsular bag is attached to the interior of the eye by the zonular ligaments.

This in situ replacement of a crystalline lens has been referred to as a phaco-ersatz procedure. U.S. Pat. No. 6,598,606 B2 to Terwee et al. discloses a method of forming an IOL in situ using a photo-curable polymerizable material, and is herein incorporated by reference in its entirety.

One drawback to the phaco-ersatz procedure described in the Terwee patent is that the shape of the lens, after creation, is not particularly controllable. That is, the shape of the lens is largely dictated by the shape of the capsular bag, and a surgeon has little control over the shape of the lens. Consequently, the newly formed lens is unlikely to provide the exact refractive power necessary to provide perfect vision. Therefore, as with a conventional IOL at least a ±1.00 diopter power correction will be required to obtain perfect vision. Furthermore, the newly formed lens will not compensate for any optical aberrations located elsewhere in the eye, such as astigmatism in the cornea.

Accordingly, there remains a need for an improved method for creating an artificial lens in situ to replace a crystalline lens.

### SUMMARY OF THE INVENTION

An object of the present invention is to provide an improved method of creating an artificial lens in situ to replace a crystalline lens.

Another object of the present invention is to provide an artificial lens that can be adjusted after being created in situ.

A further object of the present invention is to provide a method of creating an artificial lens that preserves accommodation ability.

The foregoing objects are basically obtained by an intraocular lens, including a flexible capsule adapted to be inserted into the natural lens capsular bag. A polymerized portion is positioned within the flexible capsule, and an unpolymerized material is positioned within the flexible capsule, the unpolymerized material having loose monomers and a polymerization initiator so that the unpolymerized material changes its volume when exposed to an energy source.

The foregoing objects are further obtained by an intraocular lens, including a flexible capsule adapted to be inserted into the natural lens capsular bag, the flexible capsule having a first interior chamber and a second interior chamber. An unpolymerized material is positioned in the first interior chamber, and has loose monomers and a polymerization initiator so that the unpolymerized material changes its volume when exposed to an energy source. A liquid is located in the second chamber, and is adapted to allow the flexible capsule to change shape when the natural lens focuses on a near object.

Other objects, advantages, and salient features of the present invention will become apparent from the following detailed description, which, taken in conjunction with the annexed drawings, discloses preferred embodiments of the invention.

### BRIEF DESCRIPTION OF THE DRAWINGS

Referring to the drawings which form a part of this disclosure:

FIG. 1 is a side elevational view in section taken through the center of an eye showing the cornea, pupil, crystalline lens, and capsular bag;

FIG. 2 is a side elevational view in section of the eye shown in FIG. 1 showing the capsular bag after removal of the crystalline lens;

FIG. 3 is a side elevational view in section of the eye shown in FIG. 2 showing the treatment of the interior of the capsular bag with a liquid to prevent capsular opacification;

FIG. 4 is a side elevational view in section of the eye shown in FIG. 3 showing the injection of a synthetic material with free monomers into the capsular bag using a fiber optic tube;

FIG. 5 is a side elevational view in section of the eye shown in FIG. 4 showing the removal of the fiber optic tube and curing of the injected material at the injection site to form an artificial lens;

FIG. 6 is a side elevational view in section of the eye shown in FIG. 5 showing the adjustment of the artificial lens using a laser.

FIG. 7 is a side elevational view in section of the eye shown in FIG. 5 in which the central area of the artificial lens has increased in volume in response to the application of the light;

FIG. 8 is a side elevational view in section of the eye shown in FIG. 5 in which the peripheral area of the artificial lens has increased in volume in response to the application of the light;

FIG. 9 is a side elevational view in section of the eye shown in FIG. 5 in which an anterior capsulotomy has been performed to allow the central area of the artificial lens to expand;

FIG. 10 is a side elevational view of a second embodiment of the present invention, wherein an artificial capsular bag is inserted into the natural capsular bag;

FIG. 11 is a side elevational view of a third embodiment of the present invention, wherein only the rear portion of the intraocular lens has been polymerized;

FIG. 12 is a side elevational view of the embodiment of FIG. 11 showing a portion of the intraocular lens increasing in volume when exposed to laser light;

FIG. 13 is a side elevational view of the embodiment of FIG. 11 showing a portion of the intraocular lens decreasing in volume when exposed to laser light;

FIG. 14 is a side elevational view of a fourth embodiment of the present invention, wherein the interior of the artificial bag is divided into two portions;

FIG. 15 is a side elevational view of a the embodiment of FIG. 14 showing the insertion of a liquid into one the interior chambers of the artificial bag;

FIG. 16 is a side elevational view of the embodiment of FIG. 14 showing a portion of the intraocular lens increasing in volume when exposed to laser light;

FIG. 17 is a side elevational view of the embodiment of FIG. 14 showing a portion of the intraocular lens decreasing in volume when exposed to laser light; and

FIG. 18 is a side elevational view of the embodiment of FIG. 14 showing accommodation.

### DETAILED DESCRIPTION OF THE INVENTION

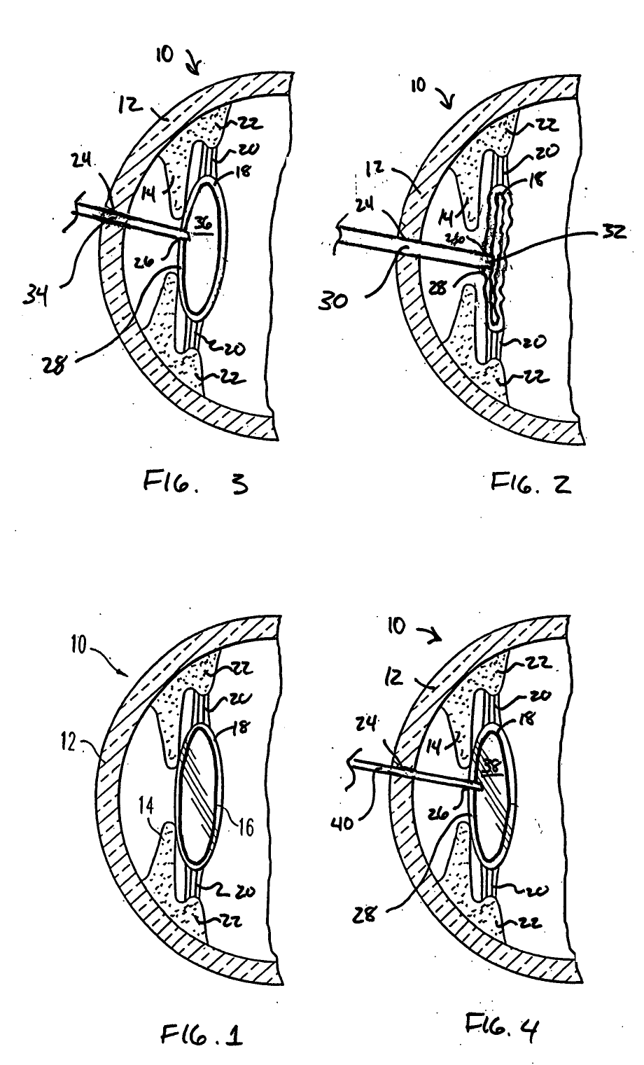

Referring initially to FIG. 1, a normal eye 10 has a cornea 12, an iris 14, and a crystalline lens 16. The crystalline lens 16 is contained within a capsular bag 18 that is supported by zonules 20. The zonules 20, in turn, are connected to the ciliary muscle 22. According to Helmholz's theory of accommodation, upon contraction of the ciliary muscle 22, the tension on the zonules 20 is released. The elasticity of the lens causes the curvature of the lens 16 to increase, thereby providing increased refractive power for near vision. Conversely, during dis-accommodation, the ciliary muscle 22 is relaxed, increasing the tension on the zonules 20 and flattening the lens 16 to provide the proper refractive power for far vision.

To replace the crystalline lens in accordance with the method of the present invention, the first step is to remove the existing lens. As illustrated in FIG. 2, the lens is removed using any technique which allows removal of the lens through a relatively small incision, preferably about a 1-2 mm incision. The preferred method is to create a relatively small incision 24 in the cornea 12 and then perform a capsulorhexis to create an opening 26 into the anterior side 28 of the capsular bag 18. An ultrasonic probe 30 is inserted into the capsular bag 18 through the opening 26. The probe's vibrating tip 32 emulsifies the lens 16 into tiny fragments that are suctioned out of the capsular bag by an attachment on the probe tip (not shown). Alternatively, the lensectomy may be performed by laser phacoemulsification or irrigation and aspiration.

Once the crystalline lens 16 has been removed, the capsular bag 18 is treated to help prevent a phenomenon known as capsular opacification. Capsular opacification is caused by the proliferated growth of the epithelial cells on the lens capsule. This growth can result in the cells covering all or a substantial portion of the front and rear surfaces of the lens capsule, which can cause the lens capsule to become cloudy and thus adversely affect the patient's vision. These cells can be removed by known techniques, such as by scraping away the epithelial cells; however, it is often difficult to remove all of the unwanted cells. Furthermore, after time, the unwanted cells will typically grow back, requiring further surgery. To prevent capsular opacification, the capsular bag 18 is treated to eliminate the proliferated growth of epithelial cells, as described below.

As seen in FIG. 3, one method of treating the epithelial cells to prevent capsular opacification is to use a cannula 34 to introduce a warm liquid 36 (preferably about <60° C.) into the capsular bag 18, filling the capsular bag 18. The liquid contains a suitable chemical that kills the remaining lens cells in the capsular bag and also cleans the interior of the capsular bag. Suitable chemicals, as well as other suitable methods of treatment that prevent capsular opacification are disclosed in U.S. Pat. No. 6,673,067 to Peyman, which is herein incorporated by reference in its entirety.

After treating the capsular bag to prevent capsular opacification, the capsular bag is filled with a synthetic, injectable material. The synthetic material is preferably a silicone based material which is un-polymerized. The material has a viscosity between about 10 centistokes (cSt) and 10,000 centistokes at body (or about 37 degrees C.) temperature so that it may be injected into the body though a cannula. The synthetic material contains loose monomers and an initiator that initiates polymerization of the loose monomers. In a preferred embodiment, the initiator is a photoinitiator so that when the material is exposed to the proper wavelength of light, preferably blue light, the initiator causes the loose monomers to polymerize. Initiators responsive to other sources of energy, such as heat or chemicals, may be used if desired.

The polymerization of the monomers caused by the initiators results in a lower concentration of monomers in the polymerized area. Through the principle of diffusion, loose monomers therefore migrate to the polymerized area, causing the polymerized area to swell. Suitable materials, and a more detailed discussion of their method of operation, are disclosed in U.S. Pat. No. 6,721,043 B2 to Platt et al., U.S. Pat. No. 6,749,632 B2 to Sandstedt et al., and U.S. Pat. App. No. 2003/0174375 A1 to Jethmalani et al, all of which are herein incorporated by reference in their entirety.

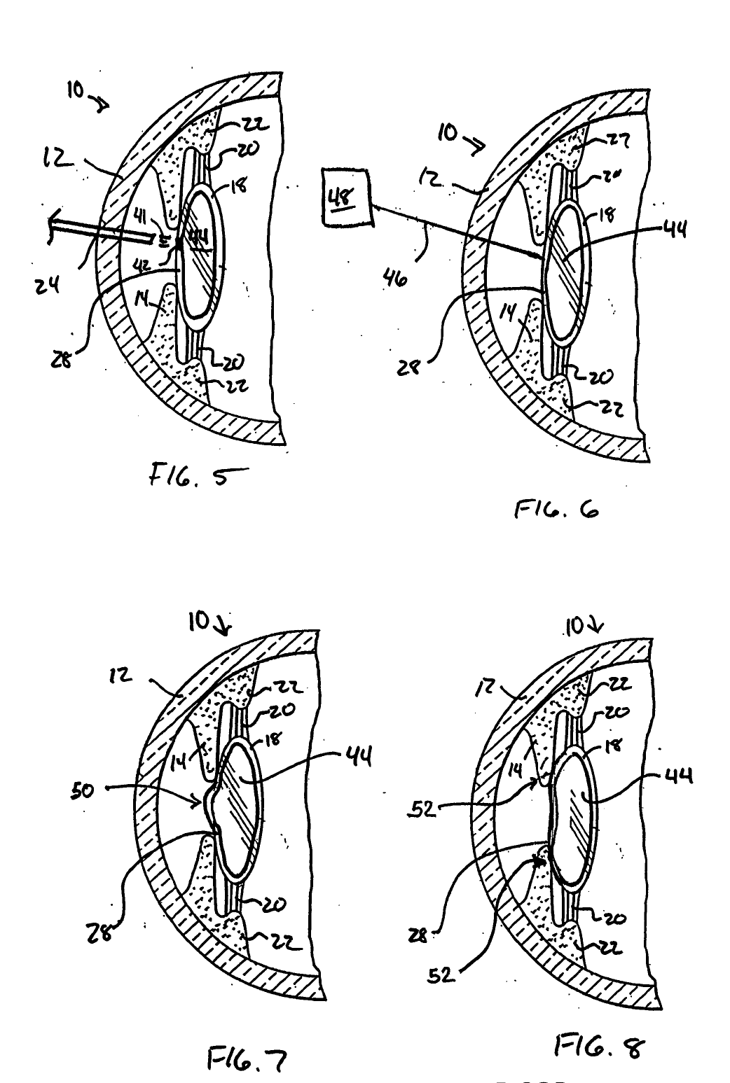

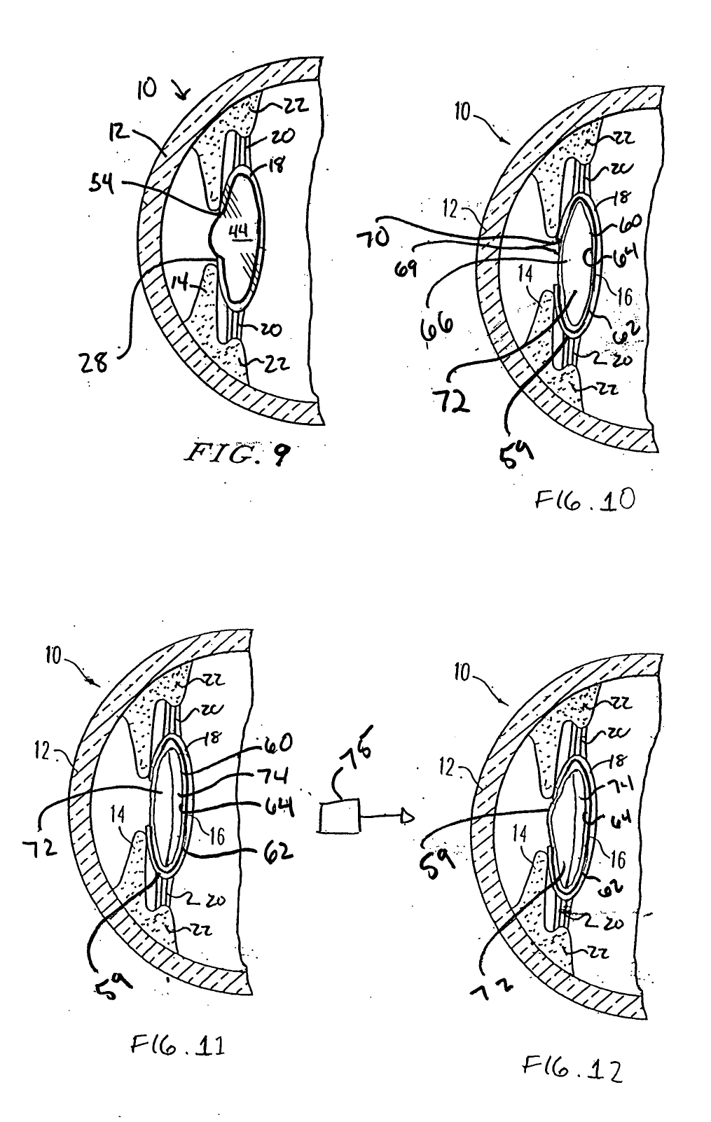

As shown in FIG. 4, the synthetic material 38 is injected into the capsular bag 18 using a hollow tube 40. Preferably, the tube 40 is a hollow fiber optic (i.e. light conducting) tube and the injection is made through the same opening 26 that was created to remove the crystalline lens 16. The amount of material that is injected into the capsular bag is chosen so that it closely approximates the desired refractive power of the original, natural lens. Any remaining fluid that is present in the capsular bag prior to injection of the synthetic material 38 can either be aspirated through another hole in the capsular bag, or can simply be allowed to leak through the edges of the capsular bag.

After the desired amount of material has been injected into the capsular bag 18, light 41 is transmitted through the light conducting tube 40 at the same time the tube is withdrawn from the opening 26 to the capsular bag 18. The light 41 is at the appropriate wavelength to initiate polymerization of the liquid material. Thus, when the tube 40 is removed, the polymerized liquid material forms a polymerized plug 42 that seals the opening 26 into the capsular bag 18, trapping the remaining liquid material inside the capsular bag. At this point, the capsular bag 18 is filled with a liquid, photo-sensitive material, thereby forming an artificial lens 44.

After creating the artificial lens 44, a suitable period of time, such as a few days, is allowed to elapse so that the eye heals and the refractive power of the eye stabilizes. The eye is then measured to determine if there are any remaining optical aberrations in the eye that need to be corrected. The eye can be measured using, for example, wavefront sensor technology. If there are any errors which need to be corrected, the artificial lens 44 can be adjusted by exposing the lens 44 to light 46, which is generated by a light source 48 (FIG. 6). Light 46 is applied in a predetermined pattern to modify the refractive properties of the lens 44 as desired to create perfect, or 20/20, far vision.

For example, referring to FIG. 7, if the surgeon determines that additional plus dioptic power is needed, the surgeon can selectively polymerize the central portion 50 of the artificial lens 44 by aiming a light with the appropriate wavelength through the cornea 12 towards the central portion 48 of the lens. As discussed above, this will cause the central portion 48 of the lens to swell, thereby providing increased plus dioptic power. Conversely, if the surgeon wishes to lower the plus dioptic power of the lens, the surgeon can direct blue light towards the periphery 52 of the lens. This will cause the periphery 52 to swell, thereby flattening the lens 44 and reducing the amount of plus dioptic power of the lens 44. Likewise, various portions of the lens may be irradiated with the light to introduce corrections for other optical aberrations, such as astigmatisms.

The adjustment process may be repeated until the desired corrective capabilities have been programmed into the lens 44. Once satisfied with the lens, the entire lens 44 is irradiated with an appropriate wavelength of light to polymerize the entire lens, thereby fixing the refractive power of the lens.

After this final polymerization of the lens, the lens 44 takes on a gel-like consistency that approximates the function of a crystalline lens. The lens 44 therefore is capable of providing accommodation. That is, in the method of the present invention, the capsular bag 18 has been left substantially intact, and the zonules 20 and ciliary muscle 22 have not been damaged. Consequently, upon contraction or relaxation of the ciliary muscle 22, the artificial lens 44 functions like a natural lens, since the polymerized material has a gel like consistency. Therefore, lens 44 can become rounder or flatter like a natural lens to provide accommodation for near vision.

Furthermore, accommodation takes place because the contraction and relaxation of the ciliary muscle 22 moves the lens forward and backward (i.e. closer to and further from the retina). This movement of the lens also produces accommodation.

FIG. 9 shows an additional method of changing the refractive power of the implanted artificial lens 44. In FIG. 9, after the lens 44 has been polymerized to a gel-like consistency, an anterior capsulotomy is performed to remove the central portion of the anterior side 28 of the capsular bag 18. This allows the gel-like lens 44 to bulge slightly forward through the capsulotomy 54 to add additional dioptic power to the lens during accommodation.

FIGS. 10-18 show an another embodiment of the present invention, wherein an IOL 59 is formed by an artificial capsular bag or capsule 60 that is positioned within the original or natural capsular bag 18.

This artificial capsular bag is formed from silicon or any other suitable transparent poymer, and is adapted to allow light within the visible spectrum to pass therethrough. Preferably, capsular bag or capsule 60 has an exterior surface 62, an interior surface 64, which defines an interior area or portion 66. Interior portion 66 can extend through the entire bag 60 or occupy a limited portion thereof. For example, portion 66 can be located in the rear portion of the bag, the front portion of the bag, the top portion of the bag, or the bottom portion of the bag. Each location of portion 66 (i.e., rear, front, top and bottom) is relative to the location of a natural human eye, and is merely used herein for ease of understanding and is not meant to limit the present invention in any manner. Additionally, portion 66 can occupy any percentage of the bag—i.e., substantially about 100% to substantially about 1%. The remainder of the bag can be filled with any suitable material, as described above, below, or in application Ser. No. 10/272,402, discussed above, or merely be defined by the thickness of the wall 68 between the exterior surface 62 and the interior surface 64.

As shown specifically in FIG. 10, the central portion 69 of the natural capsular bag along the main optical axis is removed. The artificial capsular bag 60 is then inserted into the natural capsular bag 18 through opening 70. The artificial bag 60 can be placed inside of the natural bag 18 in any manner desired. For example, bag 60 can be merely positioned within bag 18, it can be positioned in bag 18 such that bag 18 is slightly stretched, it can be positioned, such that there is a “tight” fit (i.e., the artificial bag is tightly held within the natural bag, such that there is sufficient friction that the artificial bag cannot move or only move an insubstantial amount), or the artificial lens can be positioned with the natural bag using haptics any other type of device to prevent movement thereof.

By removing the central portion 69 of the natural capsular bag to form opening 70, the natural lens along the main optical axis is removed. This eliminates or substantially eliminates the possibility of capsular opacification of the lens in this area. However, it is noted that it is not necessary to remove the portion of the capsular bag at the main optical axis, and any size opening or aperture can be formed in any portion of the natural capsular bag that enable an artificial bag to be placed therein.

The capsular bag 60 is then filled with a liquid or synthetic material 72, which preferably includes monomers and a polymerization initiator, such as a photosensitizer in the same or substantially similar manner as the method and system described above for original capsular bag 18. Material 72 does not necessarily need to include both monomers and a photosensitizer, and may include only monomers or a photosensitizer, or any other material(s) that would enable the material to polymerize and/or change shape and/or volume.

The synthetic material 72 is preferably the same of substantially similar to the materials described above or any material described in above mentioned U.S. application Ser. No. 10/272,402, the contents of which have previously been incorporated herein by reference. For example, the synthetic material 72 preferably contains loose monomers and an initiator that initiates polymerization of the loose monomers. In a preferred embodiment, the initiator is a photoinitiator so that when the material is exposed to the proper wavelength of light, preferably blue light, the initiator causes the loose monomers to polymerize. Initiators responsive to other sources of energy, such as heat or chemicals, may be used if desired.

The polymerization of the monomers caused by the initiators results in a lower concentration of monomers in the polymerized area. Through the principle of diffusion, loose monomers therefore migrate to the polymerized area, causing the polymerized area to swell. This allows the IOL to be adjusted create perfect or substantially perfect (i.e., 20/20) vision. Suitable materials, and a more detailed discussion of their method of operation, are disclosed in U.S. Pat. No. 6,721,043 B2 to Platt et al., U.S. Pat. No. 6,749,632 B2 to Sandstedt et al., and U.S. Pat. App. No. 2003/0174375 A1 to Jethmalani et al, all of which are herein incorporated by reference in their entirety.

As described in the previous embodiments, changing the volume of the IOL 59 can result in a decrease or in increase in volume, thus changing the refractive properties of the lens to increase or decrease the diopter power. Additionally, the IOL can be adjusted multiple times as described above to “fine tune” the refractive properties of the IOL. Once the IOL has the desired refractive properties, the IOL can be completely polymerized as described above.

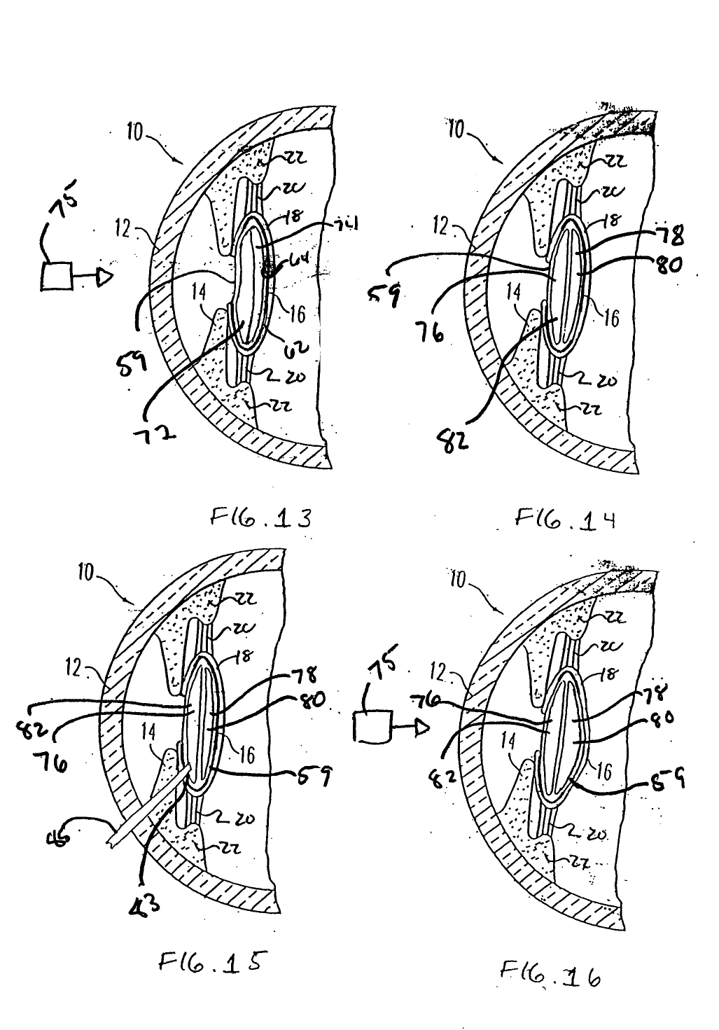

Additionally, as shown in FIG. 11, a portion 74, such as the rear portion of liquid or material 72, can be polymerized prior to insertion inside of the natural capsular bag 18. However, it is noted that the portion 74 to be polymerized does not necessarily need to be the rear portion and can be any portion desired. By polymerizing portion 74 prior to insertion into capsular bag 18, the artificial bag 60 has rigidity that can help shape and/or support the natural bag in a predetermined manner, thus facilitating the forming of the desired shape of the natural and/or artificial bags.

Furthermore, portion 74 need not necessarily be a liquid that is polymerized as discussed above, but can be a solid or substantially solid material that is generally used for forming conventional IOLs or any other suitable material. For example, portion 74 can be a separate collagen material (or any other suitable material) added to the interior or exterior of the bag or it may simply by a portion of wall between the exterior surface 62 and the interior surface 64. Additionally, the capsular bag 60 can be positioned adjacent to or coupled to a conventional IOL. For example, the capsular bag 60 can affixed to the front surface or rear surface of a conventional IOL prior to, during or after insertion of the IOL in the natural capsular bag 18.

As shown in FIGS. 12 and 13, and as discussed above, changing the volume of the front portion of the IOL 59 by exposing the unpolymerized material to a light (such as from laser 75) will result in a decrease or an increase in volume, thus changing the refractive properties of the lens to increase or decrease the diopter power. Additionally, the IOL can be adjusted multiple times as described above to “fine tune” the refractive properties of the IOL. Once the IOL has the desired refractive properties, the IOL can be completely polymerized as described above. It is noted that as with the other embodiments described above and in application Ser. No. 10/272,402, the polymerizing initiator can initiate polymerization when exposed to light, laser light, a chemical or any other suitable device and/or method.

Additionally, as shown in FIG. 14, the artificial capsular bag 60 can be divided into two interior portions, a first portion or chamber 76 and a second portion or chamber 78. Preferably, first portion 76 is located in the front part of bag 60 (i.e., closer to the anterior chamber or the iris) and second portion 78 is located in the rear or back portion of the bag (i.e., farther from the anterior chamber of iris).

Prior to insertion into the natural bag 18, the rear chamber preferably is filled with liquid or material 80, which preferably includes monomers and a polymerization initiator, such a photosensitizer in the same or substantially similar manner as the method and system described above for each of the other embodiments. Liquid 80 does not necessarily need to include both monomers and a photosensitizer, and may include only monomers or a photosensitizer, or any other material that would enable the material to polymerize and or change shape and/or volume.

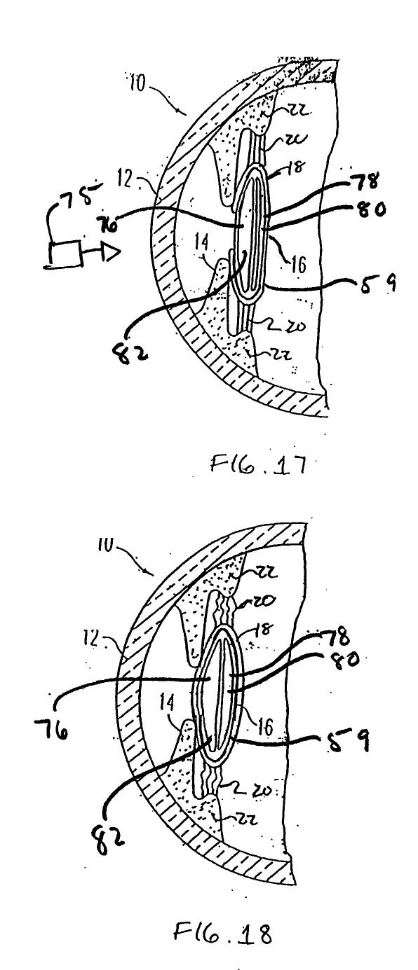

As shown in FIG. 15, the front chamber is preferably filled with a liquid polymer or material 82 suitable for insertion into the eye using a cannula 85 or any other suitable method or device. The liquid polymer can be inserted into chamber 76 through an opening 83 or a small self sealing membrane after implantation of the bag 60. It is noted that both liquid 80 and liquid 82 can be inserted into the bag at any time desired. For example, each liquid can be inserted before, after or during the surgical procedure.

It is noted that it is not necessary to fill the rear chamber with liquid 80 and the front chamber with liquid 82. This positioning of the respective liquids is merely the preferred embodiment and either of the liquids can be placed in either of the chambers. Furthermore it is noted that chambers 76 and 78 can have substantially the same volume or can have any volume desired. For example, one chamber can be larger or smaller than the other volume. Additionally, the overall volume of both chambers can occupy any amount of the volume of IOL 59 desired. For example the overall volume of chambers 76 and 78 can occupy from about 1% of the overall volume for IOL 59 to about 99%.

As shown in FIGS. 16 and 17, and as discussed above, changing the volume of the rear chamber 78 of the IOL 59 by exposing the unpolymerized material to a light (such as from laser 75) will result in a decrease or an increase in volume, thus changing the refractive properties of the lens to increase or decrease the diopter power. Additionally, the IOL can be adjusted multiple times as described above to “fine tune” the refractive properties of the IOL. Once the IOL has the desired refractive properties, the IOL can be completely polymerized as described above. It is noted that as with the other embodiments described above and in application Ser. No. 10/272,402, the polymerizing initiator can initiate polymerization when exposed to light, laser light, a chemical or any other suitable device and/or method.

As shown in FIG. 18, this embodiment allows the lens system, particularly the bag 60 to remain flexible, and thus act like a natural lens. In other words, when the eye attempts to focus on a near object (i.e., accommodate), the lens zonules loosen the natural bag, which in turn loosens the artificial bag. Each bag 18 and 60 then bulges slightly in the center. This bulging increases the refractive power of the natural lens. Conversely when the zonules tighten, each bag tends to be stretched, decreasing the refractive power. That is, when a portion of the artificial bag 60 is filled with liquid polymer 82, the artificial bag 60 and thus the natural bag 18 remain flexible after implantation. Therefore, the process of accommodation bulges the central portion of the bag, which increases the convexity of the front portion of the lens, increasing the refractive power of the lens for near vision.

Additionally, since the liquid is a polymer any exposure to light or a polymerizing agent does not polymerize the this material; however, as described above, the material 80 can be subject to exposure to different energies that would increase or decrease the volume and/or polymerize a portion or the entire volume thereof, as for any of the embodiments describe above or in application Ser. No. 10. 10/272,402.

Furthermore, the rear chamber or portion 78 can be divided into two areas or portions in a manner similar to the embodiment described in FIGS. 11-13 and FIGS. 14-18, thus forming three chambers or areas with the artificial bag 60. In this embodiment, a first portion would be filled with a material, such as liquid 82, the second portion would be filled with a material, such as material 80, and the third portion would include a polymerized material as described from FIGS. 11-13. Therefore as described above, the lens can have rigidity for insertion into the capsular bag 18 and have the volume thereof changed while inside the capsular bag to achieve the desired refractive power.

While various embodiments have been chosen to illustrate the invention, it will be understood by those skilled in the art that various changes and modifications can be made therein without departing from the scope of the invention as defined in the appended claims.

1. A method of replacing a natural lens in an eye, comprising the steps of:
removing the natural lens while leaving the capsular bag substantially intact;
removing a portion of the capsular bag along the main optical axis;
inserting an artificial bag within the capsular bag;
injecting a synthetic material into the artificial bag to form an artificial lens, the synthetic material having loose monomers and a polymerization initiator so that the synthetic material changes its volume when exposed to an energy source;
selectively exposing portions of the artificial lens to an energy source to alter the refractive properties of the artificial lens.

2. A method according to claim 1, wherein
the energy source is light.

3. A method according to claim 1, wherein
the synthetic material is injected using a fiber optic tube extending through an entrance port into the capsular bag.

4. A method according to claim 3, further comprising the step of
directing light down the fiber optic tube while withdrawing the fiber optic tube to initiate polymerization of the synthetic material and seal the entrance port to the artificial bag.

5. A method according to claim 1, further comprising the step of:
exposing substantially the entire artificial lens to an energy source to polymerize substantially all of the loose monomers, thereby fixing the refractive power of the synthetic material.

6. A method according to claim 5, further comprising the step of
performing an anterior capsulotomy to allow the central portion of the artificial lens to bulge forward during accommodation.

7. A method according to claim 1, wherein
the step of inserting an artificial bag includes inserting an artificial bag having a first internal chamber and a second internal chamber.

8. A method according to claim 7, wherein
said first internal chamber includes a polymerized material; and
said step of injecting a synthetic material into the artificial bag includes injecting said synthetic material into said second chamber.

9. A method according to claim 1, wherein
a portion of said artificial bag includes a polymerized material.

10. A method of treating an eye with a natural lens, comprising the steps of:
removing the natural lens while leaving the capsular bag substantially intact;
inserting an artificial bag into said capsular bag, said artificial bag including a front portion and rear portion;
filling the rear portion with a first substantially liquid material, first the substantially liquid material being adapted to change in volume when exposed to an energy source;
filling the front portion with a second substantially liquid material, the front portion adapted to change shape during accommodation;
measuring the eye to determine any optical aberrations; and
applying energy to the first substantially liquid material in a selective pattern to alter the refractive properties of the first substantially liquid material to correct for any optical aberrations in they eye.

11. A method according to claim 10, wherein
the front portion is filled by injecting the second substantially liquid material using a hollow tube extending through an entrance port to the artificial bag.

12. A method according to claim 11, wherein
the hollow tube conducts light; and
light is directed through the fiber optic tube while withdrawing the fiber optic tube to initiate polymerization of the synthetic material and seal the entrance port to the artificial bag

13. A method according to claim 10, wherein
the artificial bag is self sealing.

14. A method according to claim 10, further comprising the step of
exposing substantially all of the first substantially liquid material to an energy source to fix the refractive power of the material.

15. A method according to claim 10, further comprising the step of
performing an anterior capsulotomy to allow the central portion of the second substantially liquid material to bulge forward during accommodation.

16. An intraocular lens, comprising:
a flexible capsule adapted to be inserted into the natural lens capsular bag;
a polymerized portion positioned within said flexible capsule; and
an unpolymerized material positioned within said flexible capsule, and having loose monomers and a polymerization initiator so that the unpolymerized material changes its volume when exposed to an energy source.

17. An intraocular lens according to claim 16, wherein
said polymerization initiator is a photoinitiator.

18. An intraocular lens according to claim 16, wherein
said flexible capsule includes a first interior chamber and a second interior chamber.

19. An intraocular lens according to claim 18, wherein
wherein said first interior chamber is positioned in the front of the flexible capsule with respect to the eye and said second interior chamber is positioned is the rear of the flexible capsule with respect to the eye.

20. An intraocular lens according to claim 19, wherein
said polymerized portion is positioned in said second interior chamber; and
said an unpolymerized material is positioned in said first interior chamber.

21. An intraocular lens according to claim 16, wherein
said flexible capsule is adapted to be inserted into the natural lens capsular bag with haptics.

22. An intraocular lens according to claim 16, wherein
said unpolymerized material is adapted to change volume such that its diopter power increases.

23. An intraocular lens according to claim 16, wherein
said unpolymerized material is adapted to change volume such that its diopter power decreases.

24. An intraocular lens, comprising:
a flexible capsule adapted to be inserted into the natural lens capsular bag, said flexible capsule having a first interior chamber and a second interior chamber;
an unpolymerized material positioned in said first interior chamber, and having loose monomers and a polymerization initiator so that the unpolymerized material changes its volume when exposed to an energy source; and
a liquid located in said second chamber, said liquid adapted to allow the flexible capsule to change shape when the natural lens focuses on a near object.

25. An intraocular lens according to claim 24, wherein
said unpolymerized material is adapted to change volume such that its diopter power increases.

26. An intraocular lens according to claim 24, wherein
said unpolymerized material is adapted to change volume such that its diopter power decreases.

27. An intraocular lens according to claim 24, wherein
said polymerization initiator is a photoinitiator.

28. An intraocular lens according to claim 24, wherein
wherein said first interior chamber is positioned in the rear of the flexible capsule with respect to the eye and said second interior chamber is positioned is the front of the flexible capsule with respect to the eye.

29. An intraocular lens according to claim 24, wherein
said flexible capsule is adapted to be inserted into the natural lens capsular bag with haptics.

30. An intraocular lens according to claim 24, wherein
said flexible capsule third chamber; and third chamber includes a polymerized material.

31. An intraocular lens, comprising:
a flexible capsule adapted to be inserted into the natural lens capsular bag;
a polymerized portion positioned adapted to be positioned adjacent said flexible capsule when said flexible capsule is inserted into the natural lens capsular bag; and
an unpolymerized material positioned within said flexible capsule, and having loose monomers and a polymerization initiator so that the unpolymerized material changes its volume when exposed to an energy source.

32. An intraocular lens according to claim 31, wherein
said polymerization initiator is a photoinitiator.

33. An intraocular lens according to claim 31, wherein
said unpolymerized material is adapted to change volume such that its diopter power increases.

34. An intraocular lens according to claim 31, wherein
said unpolymerized material is adapted to change volume such that its diopter power decreases.
